(19) United States
(12) Patent Application Publication
(10) Pub. No.: US 2024/0347189 A1
(43) Pub. Date: Oct. 17, 2024

(54) SYSTEMS AND METHODS FOR DETERMINING CARDIAC CONTRACTILITY BASED ON SIGNALS FROM A MECHANICAL CIRCULATORY SUPPORT DEVICE

(71) Applicant: Abiomed, Inc., Danvers, MA (US)

(72) Inventors: Zhengyang Zhang, Danvers, MA (US); Govind Bhala, Danvers, MA (US); Christian Moyer, Danvers, MA (US)

(73) Assignee: Abiomed, Inc., Danvers, MA (US)

(21) Appl. No.: 18/628,505

(22) Filed: Apr. 5, 2024

Related U.S. Application Data
(60) Provisional application No. 63/494,994, filed on Apr. 7, 2023.
(60) Provisional application No. 63/495,002, filed on Apr. 7, 2023.

(51) Int. Cl. G16H 40/63; A61M 60/13; A61M 60/17; A61M 60/216; A61M 60/414; A61M 60/531; A61M 60/546; A61M 60/554; G16H 20/40; G16H 50/30

(52) U.S. Cl. CPC G16H 40/63; A61M 60/13; A61M 60/17; A61M 60/216; A61M 60/414; A61M 60/531; A61M 60/546; A61M 60/554; G16H 20/40; G16H 50/30; A61M 2205/04; A61M 2205/103; A61M 2205/3303; A61M 2205/3327; A61M 2205/3331; A61M 2205/502; A61M 2230/30

(57) ABSTRACT

Methods and apparatus for estimating a measure of cardiac contractility based on a set of features determined from a set of signals associated with a mechanical circulatory support device are provided. The method includes determining, using computer processor, a set of features based, at least in part, on the set of signals, providing the set of features as input to a machine learning model trained to output a measure of cardiac contractility, and performing an action based, at least in part, on the measure of cardiac contractility output by the machine learning model.

### CROSS REFERENCE TO RELATED APPLICATIONS

This application claims the benefit under 35 U.S.C. § 119 (c) to U.S. Provisional Patent Application No. 63/495,002, filed Apr. 7, 2023, and titled, “SYSTEMS AND METHODS FOR MEASURING NATIVE CARDIAC CONTRACTILITY USING A MACHINE LEARNING MODEL,” and claims the benefit under 35 U.S.C. § 119 (c) to U.S. Provisional Patent Application No. 63/494,994, filed Apr. 7, 2023, and titled, “SYSTEMS AND METHODS FOR MEASURING CONTRACTILITY RESERVE USING SIGNALS FROM A MECHANICAL CIRCULATORY SUPPORT DEVICE,” the entire contents of each of which is incorporated by reference herein.

### FIELD OF THE INVENTION

This disclosure relates to techniques for determining cardiac contractility based on signals from a mechanical circulatory support device.

### BACKGROUND

Cardiovascular diseases are a leading cause of morbidity, mortality, and burden on global healthcare. A variety of treatment modalities have been developed for heart health, ranging from pharmaceuticals to mechanical devices and transplantation. Mechanical cardiac support devices, such as heart pump systems, provide hemodynamic support and facilitate heart recovery. Some heart pump systems may be percutaneously inserted into the heart and can operate in parallel with the native heart to supplement cardiac output. Examples of such devices include the Impella® family of devices (Abiomed, Inc., Danvers, MA). Such heart pump systems may have sensors that detect blood pressure (or assess differential pressures across membranes) and may monitor motor current, and may use the sensor data and motor current readings to help identify pump positioning, among other things.

Such pumps can be positioned, for example, in a cardiac chamber, such as the left ventricle, to assist the heart. In this case, the pump may be inserted via a femoral artery by means of a hollow catheter and introduced up to and into the left ventricle of a patient's heart. From this position, the pump inlet may draw in blood and the pump outlet may expel the blood into the aorta. In this manner, the //heart's function may be replaced or at least assisted by operation of the pump.

A heart pump is typically connected to a respective heart pump controller that controls the heart pump, such as motor speed, and collects and displays operational data about the blood pump, such as heart signal level, battery temperature, blood flow rate and plumbing integrity. An exemplary heart pump controller is available from ABIOMED, Inc. under the trade name Automated Impella Controller®. In some instances, the controller may raise alarms when operational data values fall outside predetermined values or ranges, for example if a leak, suction, and/or pump malfunction is detected. The controller may include a video display screen upon which is displayed a graphical user interface configured to display the operational data and/or alarms.

### SUMMARY

Mechanical cardiac support devices, such as heart pump systems, provide hemodynamic support and facilitate the recovery of native heart function (e.g., native contractile function of the heart). Accordingly, having a measure of a patient's current cardiac contractility may be useful in facilitating clinical decisions regarding the patient's care while using such a device. For instance, as the patient's native cardiac contractility recovers, the patient may be weaned off the heart pump system. The inventors have recognized and appreciated that the heart pump of a heart pump system is positioned in the patient's heart in a way that provides a unique set of signals that may be used to more accurately estimate cardiac contractility for the patient compared with existing techniques for estimating cardiac contractility that do not take such signals into account. To this end, some embodiments of the present disclosure relate to improved techniques for estimating cardiac contractility using a machine learning model trained to output a measure of cardiac contractility based on a set of features determined, at least in part, on signals associated with a heart pump system.

In one aspect, a computer-implemented method is provided. The computer-implemented method includes receiving a set of signals from a mechanical circulatory support device, determining, using computer processor, a set of features based, at least in part, on the set of signals, providing the set of features as input to a machine learning model trained to output a measure of cardiac contractility, and performing an action based, at least in part, on the measure of cardiac contractility output by the machine learning model.

In another aspect, the set of signals includes at least one pressure signal and/or at least one pump function signal. In another aspect, determining the set of features comprises determining at least one feature using a combination of a first pressure signal of the at least one pressure signal and a first pump function signal of the at least one pump function signal. In another aspect, the at least one feature using a combination of a first pressure signal of the at least one pressure signal and a first pump function signal of the at least one pump function signal comprises a maximum rate of rise of left ventricular pressure during contraction. In another aspect, determining a set of features comprises, determining one or more beat-to-beat features based, at least in part, on the set of signals. In another aspect, determining the one or more beat-to-beat features comprises determining one or more of mean pump flow, mean aortic pressure, left ventricle pressure at an end of diastole, a maximum rate of rise of left ventricular pressure during contraction, or a mean motor current. In some embodiments, the beat-to-beat features may further include a maximum rate of rise of a transient signal during contraction, wherein the transient signal may include, but is not limited to, an aortic pressure signal, a motor current signal, or a combination or derivation of one or more of the aforementioned signals. In another aspect, the measure of cardiac contractility is an estimate of pre-load recruitable stroke work index. In another aspect, the machine learning model comprises a feedforward dense neural network.

In another aspect, performing an action based, at least in part, on the measure of cardiac contractility output by the machine learning model comprises displaying an indication of the measure of cardiac contractility on a user interface associated with the mechanical circulatory support device. In another aspect, the indication of the measure of cardiac contractility is a trend of cardiac contractility over a particular time range. In another aspect, performing an action based, at least in part, on the measure of cardiac contractility output by the machine learning model comprises determining a stability score for a patient in which the mechanical circulatory support device is implanted, the stability score being based, at least in part, on the measure of cardiac contractility and displaying the stability score on a user interface associated with the mechanical circulatory support device. In another aspect, performing an action based, at least in part, on the measure of cardiac contractility output by the machine learning model comprises providing a treatment recommendation for a patient in which the mechanical circulatory support device is implanted, wherein the treatment recommendation is determined based, at least in part, on the measure of cardiac contractility. In another aspect, performing an action based, at least in part, on the measure of cardiac contractility output by the machine learning model comprises adjusting an operating condition of the mechanical circulatory support device. In another aspect, adjusting an operating condition of the mechanical circulatory support device comprises adjusting a pump speed of the mechanical circulatory support device.

In another aspect, performing an action based, at least in part, on the measure of cardiac contractility output by the machine learning model comprises determining a contractility reserve based, at least in part, on the measure of cardiac contractility. In another aspect, the measure of cardiac contractility includes a first cardiac contractility output from the machine learning model based on signals from the mechanical circulatory support device at a first time and second cardiac contractility output from the machine learning model based on signals from the mechanical circulatory support device at a second time after the first time, and determining a contractility reserve comprises determining the contractility reserve based on the first cardiac contractility and the second cardiac contractility. In another aspect, determining the contractility reserve based on the first cardiac contractility and the second cardiac contractility comprises determining the contractility reserve based on a difference between the first cardiac contractility and the second cardiac contractility.

In one aspect, a controller for a mechanical circulatory support device is provided. The controller includes at least one hardware processor configured to determine a set of features based, at least in part, on a set of signals received from a mechanical circulatory support device, providing the set of features as input to a machine learning model trained to output a measure of cardiac contractility, and performing an action based, at least in part, on the measure of cardiac contractility output by the machine learning model.

In another aspect, the set of signals includes at least one pressure signal and/or at least one pump function signal. In another aspect, determining the set of features comprises determining at least one feature using a combination of a first pressure signal of the at least one pressure signal and a first pump function signal of the at least one pump function signal. In another aspect, the at least one feature using a combination of a first pressure signal of the at least one pressure signal and a first pump function signal of the at least one pump function signal comprises a maximum rate of rise of left ventricular pressure during contraction. In another aspect, the at least one hardware processor is further configured to determine the set of features by determining one or more beat-to-beat features based, at least in part, on the set of signals. In another aspect, determining the one or more beat-to-beat features comprises determining one or more of mean pump flow, mean aortic pressure, left ventricle pressure at an end of diastole, a maximum rate of rise of left ventricular pressure during contraction, or a mean motor current. In another aspect, the measure of cardiac contractility is an estimate of pre-load recruitable stroke work index. In another aspect, the machine learning model comprises a feedforward dense neural network.

In another aspect, the at least one hardware processor is configured to perform an action based, at least in part, on the measure of cardiac contractility output by the machine learning model by displaying an indication of the measure of cardiac contractility on a user interface associated with the mechanical circulatory support device. In another aspect, the indication of the measure of cardiac contractility is a trend of cardiac contractility over a particular time range. In another aspect, the at least one hardware processor is configured to perform an action based, at least in part, on the measure of cardiac contractility output by the machine learning model by determining a stability score for a patient in which the mechanical circulatory support device is implanted, the stability score being based, at least in part, on the measure of cardiac contractility, and displaying the stability score on a user interface associated with the mechanical circulatory support device. In another aspect, the at least one hardware processor is configured to perform an action based, at least in part, on the measure of cardiac contractility output by the machine learning model by providing a treatment recommendation for a patient in which the mechanical circulatory support device is implanted, and the treatment recommendation is determined based, at least in part, on the measure of cardiac contractility. In another aspect, performing an action based, at least in part, on the measure of cardiac contractility output by the machine learning model comprises adjusting an operating condition of the mechanical circulatory support device based on the measure of cardiac contractility. In another aspect, adjusting an operating condition of the mechanical circulatory support device comprises adjusting a pump speed of the mechanical circulatory support device.

In another aspect, the at least one hardware processor is configured to perform an action based, at least in part, on the measure of cardiac contractility output by the machine learning model by determining a contractility reserve based, at least in part, on the measure of cardiac contractility. In another aspect, the measure of cardiac contractility includes a first cardiac contractility output from the machine learning model based on signals from the mechanical circulatory support device at a first time and second cardiac contractility output from the machine learning model based on signals from the mechanical circulatory support device at a second time after the first time, and determining a contractility reserve comprises determining the contractility reserve based on the first cardiac contractility and the second cardiac contractility. In one aspect, determining the contractility reserve based on the first cardiac contractility and the second cardiac contractility comprises determining the contractility reserve based on a difference between the first cardiac contractility and the second cardiac contractility.

In one aspect, a heart pump system is provided. The heart pump system includes a heart pump including at least one pressure sensor configured to sense a pressure within a portion of a heart of a patient and a controller. The controller is configured to determine a set of features based, at least in part, on a set of signals received from heart pump, the set of features a first feature based on the sensed pressure, provide the set of features as input to a machine learning model trained to output a measure of cardiac contractility, and perform an action based, at least in part, on the measure of cardiac contractility output by the machine learning model.

In another aspect, the set of features further includes a second feature based on a signal corresponding to an operating state of the heart pump. In another aspect, the first feature is further based on a signal corresponding to an operating state of the heart pump. In another aspect, the first feature comprises a maximum rate of rise of left ventricular pressure during contraction. In another aspect, the controller is further configured to determine the set of features by determining one or more beat-to-beat features based, at least in part, on the set of signals. In another aspect, determining the one or more beat-to-beat features comprises determining one or more of mean pump flow, mean aortic pressure, left ventricle pressure at an end of diastole, a maximum rate of rise of left ventricular pressure during contraction, or a mean motor current. In another aspect, the measure of cardiac contractility is an estimate of pre-load recruitable stroke work index. In another aspect, the machine learning model comprises a feedforward dense neural network.

In another aspect, the heart pump system further includes a display configured to display a user interface including a representation of one or more signals associated with operation of the heart pump system, and the controller is configured to perform an action based, at least in part, on the measure of cardiac contractility output by the machine learning model by displaying an indication of the measure of cardiac contractility on the user interface. In another aspect, the indication of the measure of cardiac contractility is a trend of cardiac contractility over a particular time range.

In another aspect, the heart pump system further includes a display configured to display a user interface including a representation of one or more signals associated with operation of the heart pump system, and the controller is configured to perform an action based, at least in part, on the measure of cardiac contractility output by determining a stability score for a patient in which the heart pump is implanted, the stability score being based, at least in part, on the measure of cardiac contractility and displaying the stability score on the user interface.

In another aspect, the heart pump system further includes a display configured to display a user interface including a representation of one or more signals associated with operation of the heart pump system, and the controller is configured to perform an action based, at least in part, on the measure of cardiac contractility output by providing on the user interface a treatment recommendation for a patient in which the heart pump is implanted, wherein the treatment recommendation is determined based, at least in part, on the measure of cardiac contractility. In another aspect, the controller is configured to perform an action based, at least in part, on the measure of cardiac contractility output by the machine learning model by adjusting an operating condition of the heart pump based on the measure of cardiac contractility. In another aspect, adjusting an operating condition of the heart pump comprises adjusting a pump speed of the heart pump.

In another aspect, the controller is configured to perform an action based, at least in part, on the measure of cardiac contractility output by the machine learning model by determining a contractility reserve based, at least in part, on the measure of cardiac contractility. In another aspect, the measure of cardiac contractility includes a first cardiac contractility output from the machine learning model based on signals from the heart pump at a first time and second cardiac contractility output from the machine learning model based on signals from the heart pump at a second time after the first time, and determining a contractility reserve comprises determining the contractility reserve based on the first cardiac contractility and the second cardiac contractility. In another aspect, determining the contractility reserve based on the first cardiac contractility and the second cardiac contractility comprises determining the contractility reserve based on a difference between the first cardiac contractility and the second cardiac contractility.

### BRIEF DESCRIPTION OF DRAWINGS

FIG. 1A shows an illustrative cardiac support device that may be used with some embodiments of the present disclosure.

FIG. 1B shows an illustrative cardiac support system that includes the cardiac support device of FIG. 1A.

FIG. 2A schematically illustrates various features that may be used to estimate native cardiac contractility, in accordance with some embodiments of the present disclosure.

FIG. 2B schematically illustrates a process for using a machine learning model to estimate a measure of cardiac contractility, in accordance with some embodiments of the present disclosure.

FIG. 3A is a flowchart of a process for estimating a measure of cardiac contractility, in accordance with some embodiments of the present disclosure.

FIG. 3B is a flowchart of a process for estimating a measure of cardiac contractility using a plurality of neural networks, in accordance with some embodiments of the present disclosure.

FIG. 4 shows an example plot for deriving one or more features used to estimate a measure of cardiac contractility, in accordance with some embodiments of the present disclosure.

FIG. 5 shows an example plot comparing different estimated measures of cardiac contractility, in accordance with some embodiments of the present disclosure.

FIGS. 6A and 6B illustrate comparisons of estimated measures of cardiac contractility for loading and contractility, respectively, in accordance with some embodiments of the present disclosure.

FIG. 7 is a flowchart of a process for determining an estimate of a measure of cardiac contractility, in accordance with some embodiments of the present disclosure.

FIG. 8 shows a portion of an example user interface for displaying an estimated measure of cardiac contractility, in accordance with some embodiments of the present disclosure.

FIG. 9 shows a portion of an example user interface for displaying cardiac information for a patient having an implanted heart pump system, in accordance with some embodiments.

FIG. 10 is a flowchart of a process for determining contractility reserve, in accordance with some embodiments of the present disclosure.

### DETAILED DESCRIPTION

Contractility is a measure of cardiac function. At a cellular level, contractility may be defined as how much force a cardiac muscle cell (myocyte) can generate. At a chamber level (e.g., the left ventricle), contractility may be defined as the summation of those cellular forces acting in parallel to generate wall motion and ejection of blood. Contractility, as a property of the heart, is sometimes referred to as inotropy, cardiac inotropy, inotropic effect, or inotropic level.

Because contractility is a force measurement (e.g., measured in Newtons) at a cellular level, it may be challenging to measure the true summation of these forces. Rather, measurable proxies have been developed and validated empirically. These measures include preload-recruitable stroke work (PRSW), end systolic pressure volume relationship (ESPVR), among others. In each case, the measurable proxy may be assumed to vary with changes in contractility and, ideally, be robust (not vary) due to changes in other factors, such as loading state, heart rate, or other, non-inotropic effects. Metrics such as PRSW and ESPVR may be difficult to measure as part of clinical practice, thus other proxies such as ejection fraction, wall motion/fractional shortening, and strain-based measures (Doppler, DENSE MRI) may be used instead for estimating contractile function.

Cardiac loading (e.g., preload) is a measure of how stressed or “loaded” cardiac muscle is prior to contraction. Because cardiac muscle may automatically vary the amount of force generated depending on the myocyte length at the start of contraction, there may be a significant overlap between the concepts of contractile force, contractility, and preload. To distinguish these concepts, a force-length curve (or Frank-Starling curve, at chamber scale) may be used to denote that for a given contractile state (e.g., for a certain level of contractility), there are varying levels of force generated depending on the muscle length. It may be assumed that there is an “optimal” length at which peak force is generated. Variations along the force-length curve may be considered changes in contraction due to loading or “loading effects” and not changes in contractility. Such changes in contraction due to loading effects may also be referred to as “length-dependent activation.”

A change in contractility is a change in the peak force generated by the heart cells and heart chamber, for a given starting length. Such changes in contractility are also referred to as “length-independent activation.” Using force-length relationships, a change in contractility may be represented as a change in the entire force length curve (scaling it upwards or downwards), reflective of a new contractile state that may also vary within itself depending on the length of the muscle. Inotropic effects may be directly tied to the exchange of calcium ions (Ca++) in the myocyte. This exchange of Ca++ may be modified, for example, by altering the rate of Ca++ influx into the cells, altering the rate of release of Ca++ by the sarcoplasmic reticulum, and/or altering the sensitivity of troponin-C (that binds to C++ as part of muscle contraction cycle). At a chamber level, contractility can also be modified by altering the total number of muscle cells contributing to the “summation” of cellular contractility. For example, during disease states in which a fraction of total muscle cells are not able to cycle Ca++ efficiently, a change in contractility may be observed.

The systems, devices, and methods described herein enable a support device (e.g., a mechanical circulatory support device) residing completely or partially within an organ to assess one or more aspects of that organ's function. In particular, the systems, devices, and methods described herein enable heart pump systems, such as percutaneous ventricular assist devices, to be used to assess the function of the heart. For example, such devices may be used to estimate a measure of native cardiac contractility of a patient's myocardium.

Monitoring native cardiac contractility may be helpful for patients who are supported with a mechanical circulatory support (MCS) device as it can provide important information about the recovery of the heart muscle function. Heart recovery refers to the ability of the heart muscle to regain its function and strength after a period of injury or disease, and contractility is an important component of heart recovery, as it refers to the ability of the heart muscle to contract and pump blood effectively.

In conditions such as heart failure, myocardial infarction, or cardiomyopathy, the heart muscle may become weakened and lose its ability to contract effectively. However, with appropriate treatment and management, the heart may be able to recover its function and improve its contractility. Being able to continuously monitor and track trends of contractility may allow physicians to make informed decisions on patient management.

Some embodiments of the present disclosure relate to a device (e.g., a computing device) configured to determine native cardiac contractility (also referred to herein as simply as “contractility”) using signals from a mechanical circulatory support (MCS) device. In some embodiments, the device may be configured to use only signals from the MCS device. In some embodiments, the device may be configured to determine contractility continuously over multiple time points such that a trend of contractility over time scales of interest may be determined. In some embodiments, an amount of contractility and/or a trend of contractility may be used to determine and/or recommend a treatment strategy for a patient on mechanical circulatory device support.

The inventors have recognized and appreciated that existing techniques for measuring contractility typically rely on simultaneous measurements of left ventricular pressure and volume, and the complexity and invasiveness of this procedure often hinders its clinical adoption. Additionally, existing techniques for measuring contractility do not tend to accurately measure contractility continuously, so trends in contractility are not typically observable. To this end, some embodiments of the present disclosure relate to an improved technique for estimating contractility that uses signals associated with a pump of an MCS device during its operation.

The inventors have also recognized and appreciated that because the pump of an MCS device may be positioned in the left ventricle across the aortic valve, rate of pressure changes measured using the sensor(s) on the pump, which may reflect native cardiac systolic function, can be measured. Additionally, native heart pumping may cause the pump to deviate from its set performance curve. The amount of deviation may relate to how strong the native heart function is. In some embodiments, the rate of pressure change measurements and pump performance parameter measurements may be provided, along with other information derived from pump signals (e.g., pressure, speed, motor current), as input to a machine learning model trained to output an estimate of native cardiac contractility.

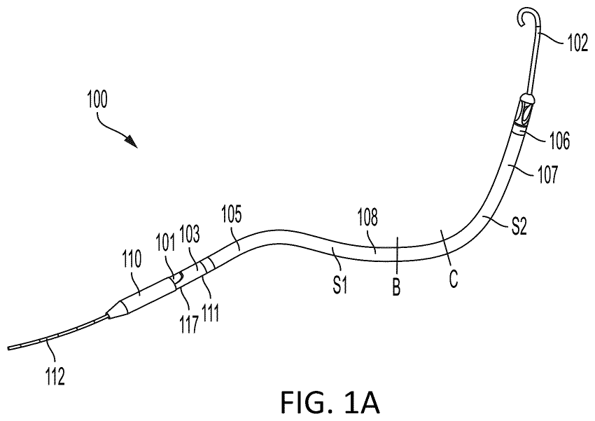

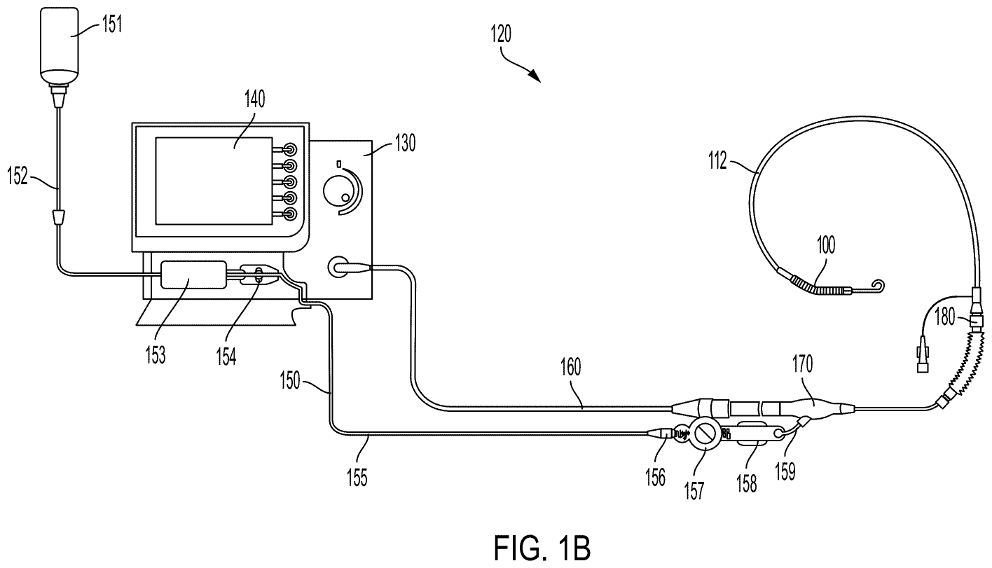

FIG. 1A shows an illustrative embodiment of a blood pump assembly 100 according to the present disclosure. The blood pump assembly 100 may include a pump 101, a pump housing 103, a proximal end 105, a distal end 107, a cannula 108, an impeller (not shown), an atraumatic extension 102, a catheter 112, an inlet area 110, an outlet area 106, and blood exhaust apertures 117. The catheter 112 may be connected to the inlet area 110 of the cannula 108 in some embodiments. The inlet area 110 may be located near the proximal end 105 of the cannula, and the outlet area 106 may be located toward the distal end 107 of the cannula 108. The inlet area 110 may include a pump housing 103 with a peripheral wall 111 extending about a rotation axis of the impeller blades, positioned radially outward of the inner surface with respect to the rotation axis of the impeller. The impeller may be rotatably coupled to the pump 101 at the inlet area 110 adjacent to the blood exhaust apertures 117 formed in the wall 111 of the pump housing 103. The pump housing 103 may be composed of a metal in accordance with some implementations. The extension 102, also referred to as a “pigtail,” may be connected to the distal end 107 of the cannula 108 and may assist with stabilizing and/or positioning the blood pump assembly 100 into the correct position in the heart. The pigtail 102 may be configurable from a straight to a partially curved configuration. The pigtail 102 may be composed, at least in part of a flexible material, and may have dual stiffness. It should be appreciated that some embodiments of the pump assembly may not include pigtail 102.

The cannula 108 may have a shape which matches (or is similar to) the anatomy of the right ventricle of a patient. In the exemplary embodiment shown in FIG. 1A, the cannula has a proximal end 105 arranged to be located near the patient's inferior vena cava, and a distal end 107 arranged to be located near the pulmonary artery. The cannula 108 may include a first segment S1 extending from the inflow area to a point B between the inlet area 110 and the outlet area 106. The cannula 108 may also include a second segment S2 extending from a point C, which is between the inlet area 110 and the outlet area 106, to the outlet area 106. In some implementations, points B and C may be located at the same location along cannula 108. The first segment S1 of the cannula may form an ‘S’ shape in a first plane. In some implementations, segment S1 can have curvatures between 30 degrees and 180 degrees. The second segment S2 of the cannula may form an ‘S’ shape in a second plane. In some implementations, segment S2 can have curvatures between 30 degrees and 180 degrees (e.g., 40°, 50°, 60°, 70°, 80°, 90°, 100°, 110°, 120°, 130°, 140°, 150°, 160°, or) 170°. The second plane can be different from the first plane. In some implementations, the second plane may be parallel or identical to the first plane.

Although shown with an ‘S’ shape, it will be appreciated that other implementations of the blood pump assembly may be formed with other shapes (e.g., a ‘U’ shape), or with no shape at all when outside the body. In such implementations, the cannula may be formed of a flexible material such that the cannula may bend during insertion and achieved the desired shape once inside the heart of the patient.

In some implementations, the blood pump assembly 100 may be inserted percutaneously through the internal jugular vein, though the right atrium and into the right ventricle. When properly positioned, the blood pump assembly 100 may deliver blood from the inlet area 110, which sits inside the patient's right atrium, through the cannula 108, to the blood exhaust apertures 117 of the pump housing 103 positioned in the pulmonary artery. Alternatively, in some implementations the blood pump assembly 100 may be inserted percutaneously through the femoral artery and into the left ventricle to deliver blood from the left ventricle into the aorta.

FIG. 1B shows that blood pump assembly 100 may form part of a cardiac support system 120. Cardiac support system 120 also may include a controller 130 (e.g., an Automated Impella Controller®, referred to herein as an “AIC,” from ABIOMED, Inc., Danvers, Mass), a display 140, a purge subsystem 150, a connector cable 160, a plug 170, and a repositioning unit 180. As shown, controller 130 may include display 140. Controller 130 may be configured to monitor and control operation of blood pump assembly 100. During operation, purge subsystem 150 may be configured to deliver a purge fluid to blood pump assembly 100 through catheter 112 to prevent blood from entering the motor (not shown) of the heart pump. In some implementations, the purge fluid is a dextrose solution (e.g., 5% dextrose in water with 25 or 50 IU/mL of heparin, although the solution need not include heparin in all embodiments). Connector cable 160 may provide an electrical connection between blood pump assembly 100 and controller 130. Plug 170 may connect catheter 112, purge subsystem 150, and connector cable 160. In some implementations, plug 170 includes a storage device (e.g., a memory) configured to store, for example, operating parameters to facilitate transfer of the patient to another controller if needed. Repositioning unit 180 may be used to reposition blood pump assembly 100 in the patient's heart (e.g., by holding a position of the pump assembly relative to the patient).

As shown in FIG. 1B, in some embodiments, the cardiac support system 120 may include a purge subsystem 150 having a container 151, a supply line 152, a purge cassette 153, a purge disc 154, purge tubing 155, a check valve 156, a pressure reservoir 157, an infusion filter 158, and a sidearm 159. Container 151 may, for example, be a bag or a bottle. As will be appreciated, in other embodiments the cardiac support system 120 may not include a purge subsystem. In some embodiments, a purge fluid may be stored in container 151. Supply line 152 may provide a fluidic connection between container 151 and purge cassette 153. Purge cassette 153 may control how the purge fluid in container 151 is delivered to blood pump assembly 100. For example, purge cassette 153 may include one or more valves for controlling a pressure and/or flow rate of the purge fluid. Purge disc 154 may include one or more pressure and/or flow sensors for measuring a pressure and/or flow rate of the purge fluid. As shown, controller 130 may include purge cassette 153 and purge disc 154. Purge tubing 155 may provide a fluidic connection between purge disc 154 and check valve 156. Pressure reservoir 157 may provide additional filling volume during a purge fluid change. In some implementations, pressure reservoir 157 may include a flexible rubber diaphragm that provides the additional filling volume by means of an expansion chamber. Infusion filter 158 may help prevent bacterial contamination and air from entering catheter 112. Sidearm 159 may provide a fluidic connection between infusion filter 158 and plug 170. Although shown as having separate purge tubing and connector cable, it will be appreciated that in some embodiments, the cardiac support system 120 may include a single connector with both fluidic and electric lines connectable to the controller 130.

During operation, controller 130 may be configured to receive measurements from one or more pressure sensors (not shown) included as a portion of blood pump assembly 100 and purge disc 154. Controller 130 may also be configured to control operation of the motor (not shown) of the blood pump assembly 100 and purge cassette 153. In some embodiments, controller 130 may be configured to control and measure a pressure and/or flow rate of a purge fluid via purge cassette 153 and purge disc 154. During operation, after exiting purge subsystem 150 through sidearm 159, the purge fluid may be channeled through purge lumens (not shown) within catheter 112 and plug 170. Sensor cables (not shown) within catheter 112, connector cable 160, and plug 170 may provide an electrical connection between components of the blood pump assembly 100 (e.g., one or more pressure sensors) and controller 130. Motor cables (not shown) within catheter 112, connector cable 160, and plug 170 may provide an electrical connection between the motor of the blood pump assembly 100 and controller 130. During operation, controller 130 may be configured to receive measurements from one or more pressure sensors of the blood pump assembly 100 through the sensor cables (e.g., optical fibers) and to control the electrical power delivered to the motor of the blood pump assembly 100 through the motor cables. By controlling the power delivered to the motor of the blood pump assembly 100, controller 130 may be operable to control the speed of the motor.

Various modifications can be made to cardiac support system 120 and one or more of its components. For instance, one or more additional sensors may be added to blood pump assembly 100. In another example, a signal generator may be added to blood pump assembly 100 to generate a signal indicative of the rotational speed of the motor of the blood pump assembly 100. As another example, one or more components of cardiac support system 120 may be separated. For instance, display 140 may be incorporated into another device in communication with controller 130 (e.g., wirelessly or through one or more electrical cables).

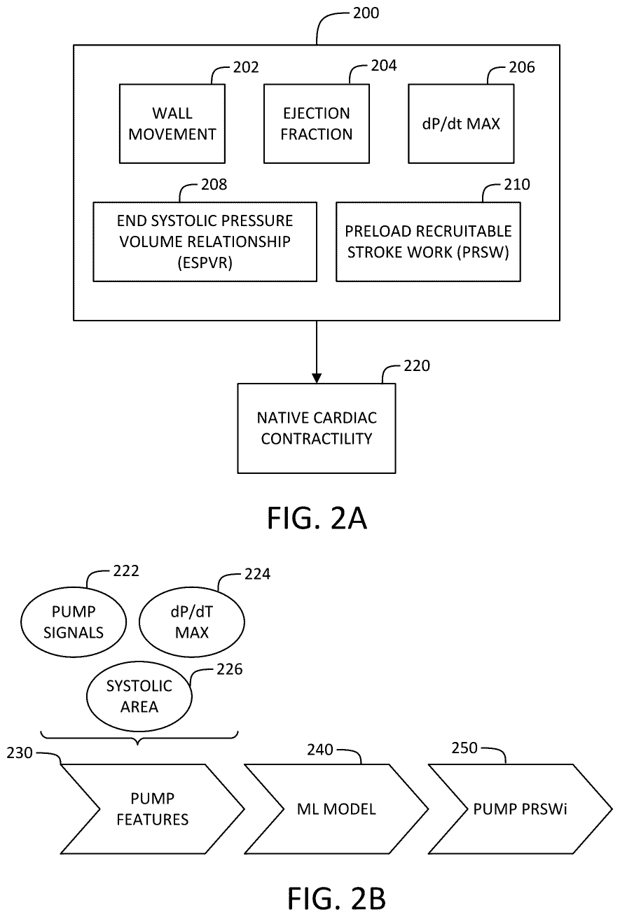

As described herein, signals from an MCS device (e.g., blood pump assembly 100) may be used to estimate native cardiac contractility. In particular, a controller of an MCS device may determine pressure signals (e.g., left ventricular pressure) that be used, at least in part, to estimate or approximate one or more measures of cardiac contractility, in accordance with some embodiments of the present disclosure. FIG. 2A schematically illustrates various features 200 that may be used to estimate native cardiac contractility 220 in accordance with some embodiments of the present disclosure. As described herein, because contractility may not be capable of being measured directly, features 200 may include one or more contractility proxy features (examples of which are described herein) such as wall movement 202, ejection fraction 204, end systolic pressure volume relationship (ESPVR) and preload recruitable stroke work (PRSW). Some contractility proxy features (e.g., ESPVR and PRSW) have been shown to be more representative of native cardiac contractility measured, for example, in animal studies, when compared with other contractility proxy features (e.g., ejection fraction). As shown in FIG. 2A, features 200 may also include information determined based on signals (e.g., pressure signals) associated with operation of a heart pump. For instance, one or more features may be derived from the LV pressure signal including, but not limited to, the maximum slope of the signal during contraction (dP/dt max 206) or the slope of the signal during contraction normalized to a pressure. In some embodiments, features 200 may include a normalized feature. For instance, features 200 may include dP/dt normalized to the mean pressure, normalized to the pressure at end-diastole prior to start of contraction, or normalized to the integral of the pressure during the contractile period up to the maximum dP/dt point. Features 200 may be used alone or combined in any suitable way to estimate native cardiac contractility, examples of which are described herein.

The inventors have recognized and appreciated that some features (e.g., features 200) do not, when considered in isolation, provide the best estimate native cardiac contractility. Accordingly, some embodiments of the present disclosure relate to processing MCS device signals and/or features derived from MCS device signals using a machine learning approach to empirically arrive at an improved estimated measure of cardiac contractility. FIG. 2B, schematically illustrates how a machine learning model may be used to estimate cardiac contractility, in accordance with some embodiments of the present disclosure. As shown in FIG. 2B, one or more pump features 230, examples of which include, but are not limited to, features determined from pump signals (e.g., motor current, pressure, pump flow rate) 222, features determined from pressure waveform signals (e.g., dP/dt max) 224, and correlation features (e.g., systolic area) 226 may be provided as input to a machine learning (ML) model 240 trained to output an estimated measure of cardiac contractility 250 based on signals associated with the pump of an MCS device. In the example of FIG. 2B, the estimated measure of cardiac contractility 250 is pump preload recruitable stroke work index (pump PRSWi).

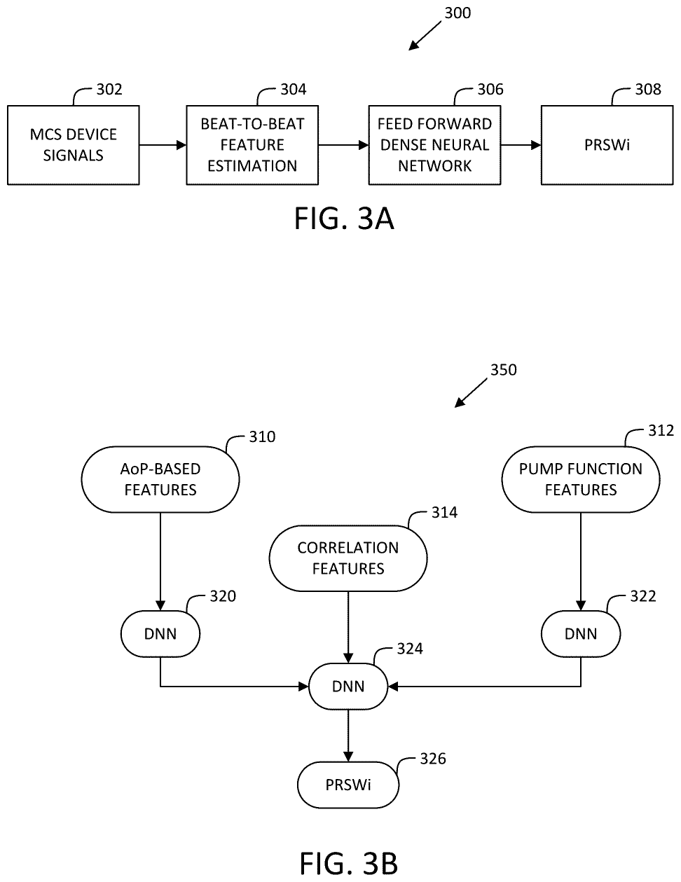

FIG. 3A is flowchart of a process 300 for determining an estimated measure of cardiac contractility (e.g., pump PRSWi) using a machine learning model, in accordance with some embodiments. Although pump PRSWi is described as the estimated measure of cardiac contractility herein, it should be appreciated that a machine learning model may alternatively be trained to output an estimate of other proxies of cardiac contractility, such as ESPVR. In act 302, one or more MCS device signals (e.g., pressure signals, motor current, blood flow rate) are received. Process 300 may then proceed to act 304, where one or more beat-to-beat features are estimated based, at least in part, on the received MCS device signal(s). Non-limiting examples of beat-to-beat features that may be estimated in act 304 include mean pump flow between two consecutive beats, mean aortic pressure between two consecutive beats, left ventricle (LV) pressure at the end of diastole (LVED), dP/dt max defined as the maximum rate of rise of LV pressure during LV contraction, and mean motor current of the pump between two consecutive beats. Process 300 may then proceed to act 306, where one or more of the beat-to-beat features estimated in act 304 are provided as input to a trained machine learning model. In the example process 300, the trained machine learning model may be a feedforward dense neural network. Process 300 may then proceed to act 308, where an estimated measure of cardiac contractility is output from the trained machine learning model. In the example process 300, the output of the trained machine learning model is pump PRSWi.

It should be appreciated that any suitable model architecture may be used for the trained machine learning model, in accordance with some embodiments of the present disclosure. An example architecture of a ML model 350 that may be used in accordance with some embodiments of the present disclosure is shown in FIG. 3B. In the example shown in FIG. 3B, the ML model 350 is implemented using a deep neural network (DNN) architecture, although it should be appreciated that other ML network architectures may alternatively be used. Additionally, ML model 350 is illustrated in FIG. 3B as including three DNNs (DNNs 320, 322, and 324). It should be appreciated that the use of three DNNs is merely exemplary and any number of DNNs including, but not limited to a single DNN or more than three DNNs may alternatively be used in accordance with embodiments of the present disclosure.

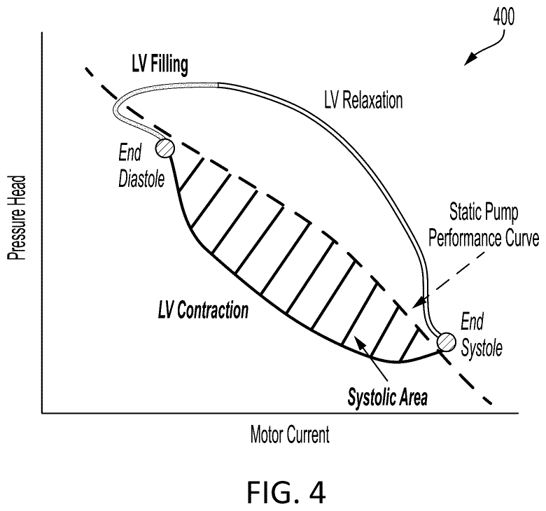

As shown in FIG. 3B, the ML model 350 receives as input AoP-based features 310, pump function features 312, and correlation features 314. AoP-based features 310 may include features associated with pressure signals measured by or derived from pressure sensors located on a pump of an MCS device. For example, AoP-based features 310 may include systolic pressure measurements, diastolic pressure measurements, time-based pressure measurements (e.g., dP/dt max), or any other suitable pressure measurements associated with a pressure signal (e.g., an LV pressure signal). Pump function features 312 may include features associated with the operation of the pump itself, examples of which include, but are not limited to, motor current, pump speed, and pump flow rate. Correlation features 314 may include features that depend, at least in part, on AoP-based features 310 and pump function features 312. For instance, FIG. 4 shows a plot 400 of LV pressure (y-axis) vs. motor current (x-axis). Values of features derived from such a relationship, an example of which includes systolic area as shown in FIG. 4, may be included in correlation features 314. Each of the AoP-based features 310, pump function features 312 and correlation features 314 may be provided as input to ML model 350 that, when trained (e.g., using pre-clinical data), may be configured to output a value for PRSWi 326, as an estimated measure of cardiac contractility.

Although ML model 350 is shown in FIG. 3B as receiving three different types of inputs (i.e., AoP-based features 310, pump function features 312, and correlation features 314), it should be appreciated that some embodiments may include fewer (e.g., one or two) or additional (e.g., more than three) types of features as input to ML model 350, and embodiments of the present disclosure are not limited in this respect. Additionally, within each of the different types of features provided as input, any suitable number of features may be used. For example, in one embodiment, seven AoP-based features 310, three pump function features 312, and nine correlation features 314 may be used. In another example, five AoP-based features 310, six pump function features 312, and seven correlation features 314 may be used.

In some embodiments, features provided as input to ML model 350 may be categorized and provided to separate network components (e.g., separate DNNs) prior to being combined to determine an estimated measure of cardiac contractility (e.g., pump PRSWi). Such an approach may facilitate use of a simpler and/or more compact ML model that can be implemented on a processor with limited processing resources (e.g., on the controller of the heart pump system). In some embodiments, it may not be necessary to categorize the features prior to processing them using an ML model. For instance, a set of features (e.g., beat-to-beat features) determined from the MCS device signals may be provided as input to a trained neural network without categorization, and the network may over time learn how to weight different features to determine a best estimated measure of cardiac contractility.

In some embodiments, ML model 350 may be trained using a leave one out training process. For instance, multiple ML models may be defined, each with the same architecture and hyperparameters. Each of the multiple ML models may be trained on a separate dataset of features and a final ML model may be defined and trained based on one or more of the multiple ML models.

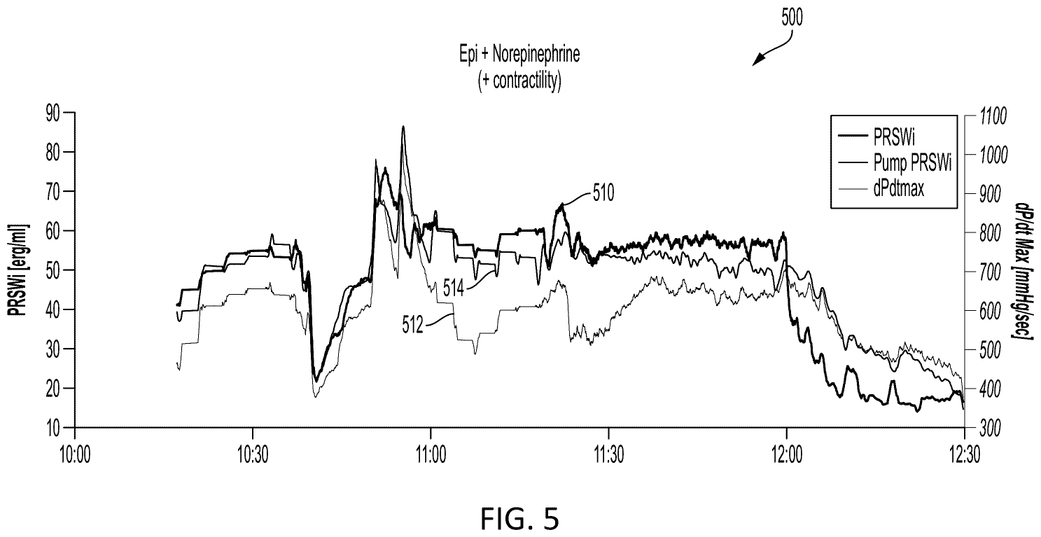

In some embodiments, the estimated measure of native cardiac contractility as determined based on the output of the ML model may be visualized, trended, and/or tracked longitudinally. For instance, an estimated contractility value may be displayed on one or more displays associated with an MCS device. FIG. 5 shows an example plot 500 of an estimated measure of cardiac contractility (Pump PRSWi) 514 determined using a trained machine learning model, as described herein. The Pump PRSWi value 514 is trended over a time of hours relative and is compared to a measured (e.g., ground truth) PRSWi value 510 (e.g., when a stress test is performed), and a measured dP/dt max value 512 determined based on the LV pressure signal of a heart pump system. As can be observed in FIG. 5, the Pump PRSWi value 514 may more closely track the measured PRSWi value 510 compared with the dP/dt max metric 512 alone, illustrating the effectiveness of using ML-based techniques to reliably estimate cardiac contractility. For example, when an increase in contractility is induced around the 10:50 mark of plot 500 there is a good correspondence between the Pump PRSWi metric 514 and the other PRSW metrics. Furthermore, plot 500 shows that following the initial induction of contractility (e.g., after the 11:00 mark of plot 500), the Pump PRSWi metric 514 more closely tracks the ground truth PRSWi metric 510 compared with the dP/dt max metric 512. Such comparisons demonstrate the ability of the trained ML model to accurately predict native cardiac contractility based on signals associated with the pump of the MCS device.

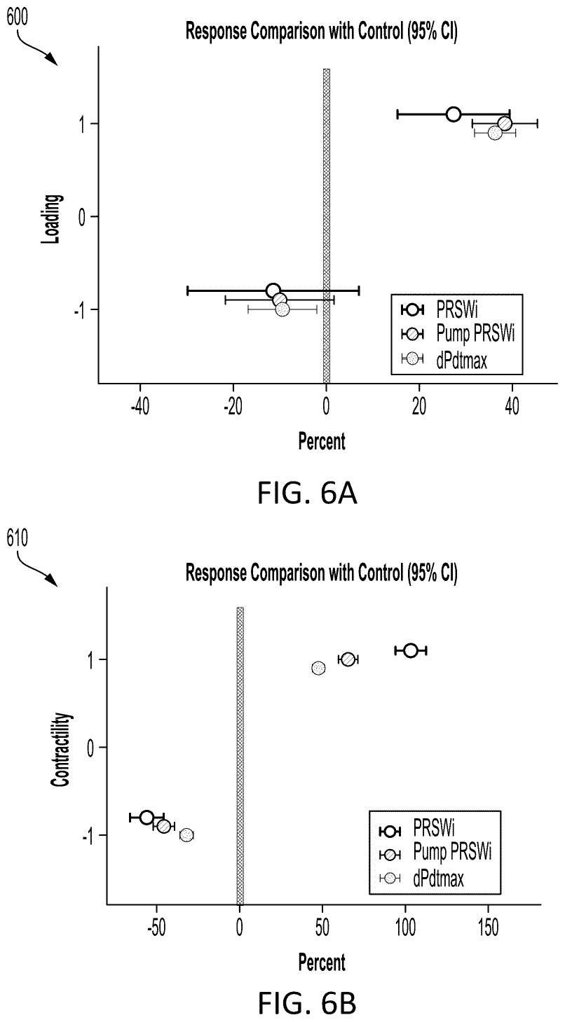

FIGS. 6A and 6B further illustrate a comparison of the pump PRSWi value determined as output of a trained ML model using the techniques described herein compared with proxy metrics conventionally used to estimate contractility. Ideally, an accurate estimated measure of cardiac contractility would be sensitive to changes in contractility and insensitive to changes in loading. Plot 600 in FIG. 6A and plot 610 in FIG. 6B show that the pump metric performance (pump PRSWi) determined using the techniques described herein is located between values for a reference (PRSWi) and an input to the model (dP/dt max) in terms of sensitivity to both loading and contractility, demonstrating that the pump-signal based techniques described herein for determining contractility perform similarly with existing techniques for determining contractility.

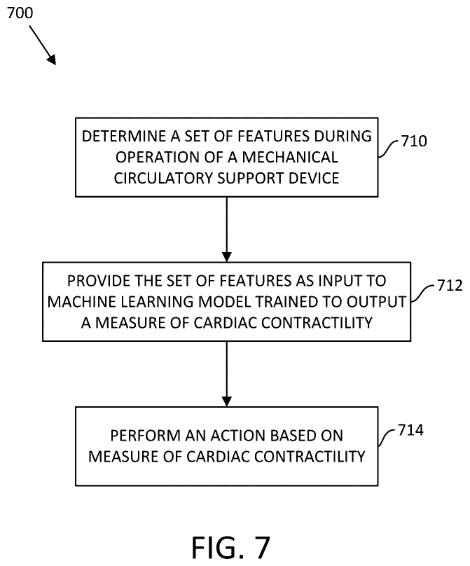

FIG. 7 illustrates a process 700 for estimating a measure of cardiac contractility using signals from a mechanical circulatory support (MCS) device and a trained machined learning model, in accordance with some embodiments of the disclosure. Process 700 may start in act 710, where a set of features during operation of a MCS device are determined. For example, as described herein, one or more signals associated with operation of an MCS device (e.g., pressure signals, flow rate signals, motor current signals, etc.) may be processed to extract one or more features (e.g., one or more beat-to-beat features) that may be included in the set of features. Process 700 may then proceed to act 712, where the set of features is provided as input to a machine learning ML) model trained to output a measure of cardiac contractility. For example, as described herein, an ML model may be trained (e.g., using pre-clinical data) to estimate PRSWi based on features extracted from pump signal data.

Process 700 may then proceed to act 714, where an action is performed based, at least in part, on the measure of cardiac contractility output from the ML model. In some embodiments, the action may be to display a value and/or a trend of the cardiac contractility measurement on a user interface associated with the MCS device. In some embodiments, the cardiac contractility measurement may be measured over time compared to a baseline measurement at a prior point in time or as an absolute or relative (e.g., percentage) change in contractility from the baseline measurement. In such embodiments, an indication of the change in contractility relative to the baseline measurement may be displayed. In some embodiments, the action may be to provide clinical decision support (CDS) to a user (e.g., a physician or other healthcare professional) associated with the MCS device. For instance, when the cardiac contractility metric is above (or below) a threshold value, an alert or other indication may be provided to the user, which may indicate to the user that the patient's cardiac function is improving (or declining) and the user may take appropriate action to improve patient management based on the alert/indication. In some embodiments, the action may be to use the cardiac contractility measurement to modify one or more other metrics that may be used to facilitate patient management. In some embodiments, the action may be to adjust (with or without user assistance) an operating condition of the MCS device. For example, a pump speed of the MCS device may be adjusted based, at least in part, on the measure of cardiac contractility. In this way, the measure of cardiac contractility may be used as feedback to adjust the support provided by the MCS device according to the patient's needs to further encourage the patient's recovery of native heart function.

In some embodiments, once an MCS device is deployed, the measure of cardiac contractility (e.g., PRSWi) parameter may be continuously available for use and trending, cither via user initiation or automatic system initiation. The contractility measure may be used for broader clinical decision support (CDS) algorithms for patient management on an MCS device, such as device escalation, device weaning, device support titration, secondary device titration, ventilator titration/extubation, or pharmacological titration of vasoactive or inotropic medication.

The contractility metric may be visualized, trended, tracked longitudinally over daily/weekly basis as a metric of native heart recovery. This measure may have significant short- and/or long-term prognostic power on patient outcomes.

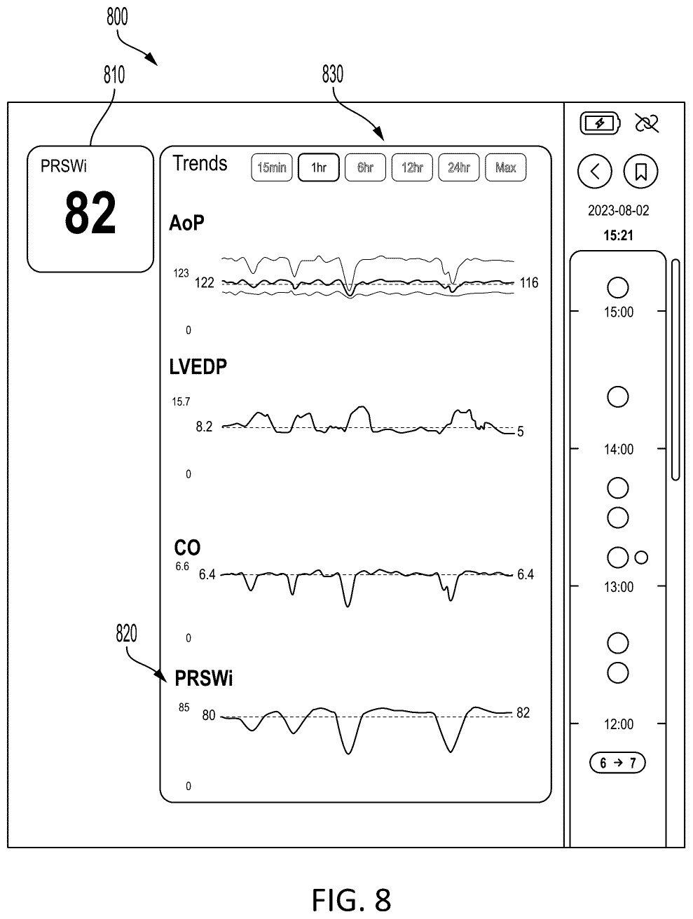

FIG. 8 shows an example of a portion of a user interface 800 in which an estimated measure of cardiac contractility may be displayed, in accordance with some embodiments. As shown in FIG. 8, the estimated measure of cardiac contractility may be shown as a numerical value 810 and/or as a trend 820 over time. In some embodiments, user interface 800 may include a time window selector 830 configured to enable a user to interact with the user interface to show the trends (e.g., trend 820) over different timescales.

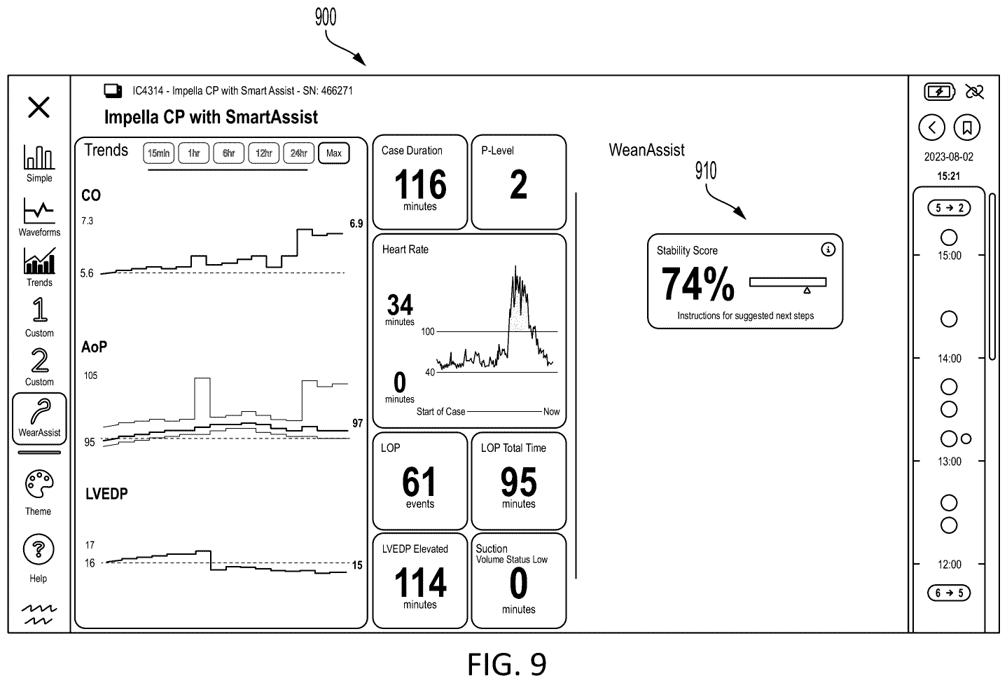

As described herein, in some embodiments, the estimated cardiac contractility measurement may be used to modify one or more other metrics determined based on signals associated with an MCS device to provide improved patient management. For some patients having an implanted MCS device, it may be desirable to wean the patient off the device (and possibly eventually explant the device) as the patient's native heart function recovers. The recovery of native heart function may be characterized in some instances, as the recovery of contractile function of the heart. Accordingly, in some embodiments, the estimated measure of contractility may be used, at least in part, to provide guidance to a clinician about how and/or when to wean a patient off of an MCS device. FIG. 9 shows an example of a portion of a user interface 900 for a weaning assist clinical decision support tool, in accordance with some embodiments of the present disclosure. In the example user interface 900 shown in FIG. 9, the weaning assist tool includes a stability score 910, which may indicate the relative stability of the patient as determined by a plurality of variables or metrics associated with the MCS device. In some embodiments, at least one of the metrics used to determine the stability score 910 may be the estimated cardiac contractility metric (e.g., pump PRSWi). Based on the stability score 910 of the patient, a healthcare provider may decide to modify the amount of support provided to the patient by the MCS device with a goal of weaning the patient off the device or providing additional support if warranted.

The concept of contractility reserve may be defined as the “extra capacity” or “reserve” that a heart has available to it to increase contraction in response to a change in physiologic state. For instance, following an acute event (e.g., an acute myocardial infarction (AMI)), there may be a decrease in contractility. In response, the heart may, in part, increase contractility in the remaining available muscle cells to preserve afterload/output/end organ perfusion. Similarly, in an event such as bleeding/hemorrhage, a decrease in mean blood pressure may trigger the heart to respond to the event, in part, by increasing contractility to preserve perfusion. Adding to the complexity, these changes are often overlaid with other response mechanisms, including the changes in loading, changes in heart rate (chronotropy), and/or changes in vascular properties (compliance and resistance), all of which tend to alter the hemodynamic state in parallel with true changes in inotropy.

To isolate and measure contractility reserve, a “cardiac stress test” is typically performed, either using exercise or pharmacologic agents to trigger a change in inotropy. In a typical stress test, the heart is challenged (physically or pharmacologically) to induce changes in cellular Ca++ handling, which results in a change in inotropy. The heart may be repeatedly challenged to push the heart to a “maximum” inotropic state, which may represent the highest possible contractility (zero remaining reserve). The total contractility reserve can then be measured by subtracting the peak state contractility from the baseline state contractility, with the difference being the “contractility reserve.” The contractility reserve can be normalized to the baseline state or other factors. Alternatively, a ratio of the contractility metric at peak state to baseline state can be used to quantify contractility reserve on a normalized basis. Some typical clinical measures of contractility reserve include change in ejection fraction or change in “wall motion score index” (WMSI). Other measures of contractility reserve may also use imaging based left ventricle (LV) dimensions changes or LV global strain (e.g., circumferential or longitudinal).

Measures of contractility reserve have shown a strong correlation with clinical outcome, especially long-term recovery following an acute event. The concept of “native heart recovery” may be intrinsically tied to the recovery of contractility reserve. Accordingly, it may be important to quantify contractility reserve in patients where recovery of native heart function is a treatment goal of an inpatient stay. In some instances, knowledge of contractility reserve may be used as a marker in decisions to wean/explant a support device (e.g., a mechanical circulatory support device), reduce pharmacological support, and/or discharge a patient from a hospital. Similarly, measurements of contractility reserve may be a useful indicator of a patient's quality of life handling future, stress states (e.g., walking up stairs), that may not be easily assessable when a patient is at rest in a hospital bed.

Contractility reserve has been shown to be a valuable prognostic indicator of short and long term outcome for a number of different cardiac diseases. Accordingly, the inventors have recognized and appreciated that measuring, estimating, and/or predicting contractility reserve may be a valuable tool for patient management of patients receiving mechanical circulatory support. The inventors have further recognized and appreciated that existing solutions for determining contractility reserve typically do not measure contractility accurately. Thus, estimates of contractility reserve based on such inaccurate contractility measurements are likely to have compounded error, which may limit clinical utility of such measures. Additionally, existing techniques typically require intervention (e.g., a stress test) to induce a change in a patient's cardiac state and to measure differences in contractility when the patient has different cardiac states. Limitations of such techniques may include that they cannot be performed continuously, and in some instances may not be able to be performed at all (e.g., if a patient cannot tolerate a stress event and/or if there are time/resource limitations). Further, existing techniques may not use signals such as LV pressure or LV volume that may be most closely associated with changes in true cellular contractility.

The inventors have recognized and appreciated that the techniques described herein for estimating native cardiac contractility using signals from a mechanical circulatory device may also be useful in estimating contractility reserve. For instance, cardiac contractility may be measured at multiple points (or windows) in time using the techniques described herein. The multiple measurements may then be used to estimate contractility reserve for the patient. For example, a difference in contractility measured at two different points in time may be used to estimate a patient's contractility reserve. In some embodiments, rather than subjecting a patient to a traditional cardiac stress test (e.g., induced by exercise or a pharmaceutical agent), the operation of the heart pump itself may be used to apply different stresses on the patient's heart to measure cardiac reserve. For instance, changes in pump function may induce changes in contractility as a “mini stress test.” In some embodiments, the pump may be configured to operate continuously and predictions of cardiac reserve may be assessed or trended over time using one or more of the techniques described herein.

In some embodiments, trending contractility may be determined during a typical clinical stress test. For instance, a device may queue or store a first contractility value (e.g., using the techniques described herein) determined before the stress test, execute the stress test (e.g., using a pharmaceutical such as dobutamine or using exercise), and store a second contractility value measured at peak stress during the stress test. The first and second contractility values may be used to determine the patient's contractility reserve. For instance, the contractility reserve may be determined based on a ratio of the first and second contractility values, a difference between the first and second contractility values, a percent difference between the first and second contractility values, or a regression fit/projection of peak stress if multiple stress points are collected.

In some embodiments, a small change in contractility may be induced in a patient such that the degree of change correlates to the true (or near true) contractility reserve of the patient according to a known transformation. Changes in the pump speed of a mechanical circulatory support device over short time periods (e.g., ˜1 minute time periods) may induce a change in coronary perfusion pressure, leading to a change in coronary flow, and a temporary change in contractility, which may trigger the reserve action (to a certain degree), to recover homeostasis, with the degree of change correlating to peak reserve. The coronary perfusion pressure may represent the difference between the aortic pressure and the left ventricular pressure. Different pump flows may change coronary flow/perfusion, which may mimic or otherwise approximate a Dipyridamole stress test. The runtime at high and low flows may be longer to allow the heart to react to the change in coronary flow, and changes in systolic and diastolic functions may be mapped to contractility reserve. In some embodiments, a pressure measurement (e.g., reflecting coronary perfusion pressure) from the heart pump may be used to estimate contractility reserve when the heart transitions from a first “unstressed” state to a “stressed” state.

In some embodiments, contractility reserve may be determined based, at least in part, on electrocardiogram (ECG) information combined with information from a left ventricular (LV) pressure waveform. For instance, an ECG-based lead signal may be used to measure a component of excitation-contraction coupling (EC-Coupling), such as a delay between the start of electrical contraction and mechanical upstroke, which may reflect excess capacity of calcium channels. One or more extracted features of an ECG waveform (e.g., QRS interval, P-R interval, T wave amplitude, morphology of electrical depolarization phase and repolarization phase, etc.) may be combined with information associated with the profile of LV pressure measured from a pump of a mechanical circulatory support device to predict contractility reserve for a patient.

In some embodiments, contractility reserve may be determined based, at least in part, on known LV end systolic volume (LV ESV) combined with information from a LV pressure waveform (e.g., LV SP/LV ESV). Additionally or alternatively to using ECG information, information from other sources (e.g., first and second heart sounds) that contain information about turbulence/extent of initial contraction may be used. When combined with one or more features of the LV pressure signal, the relationship between heart sound and pressure generation may indicate an expected “hidden reserve.” For instance, the LV pressure waveform may reflect the extent of contraction (force) and the heart sound may reflect the extent of motion (strain/shortening/valve action).

In some embodiments, contractility reserve may be determined based, at least in part, on morphological signals in available waveforms including, but not limited to, a LV diastolic filling curve, current contractility value, heart rate, cardiac output, and/or cardiac power output (CPO). In such embodiments, the focus may be on lusitropy/relaxation during early diastole and the extent of true passive period during diastole (fast lusitropy/short relaxation time), which may correlate to more contractility reserve capacity. When the heart is operating at or near capacity (lower reserve), it may be likely that there is poor/no relaxation time and little true passive period.

In some embodiments, contractility reserve may be determined based, at least in part, using a system (e.g., all available signals) that implements a lumped parameter model of circulation and the heart. In this embodiment, a stress test may then be simulated using the model and contractility reserve may be estimated from the simulation. The contractility reserve may be determined based on a change in true contractility (e.g., PRSW) or via another simulated proxy. For instance, an estimate of cardiac contractility (e.g., pump PRSWi) using the techniques described herein may be determined at multiple points in time, and the contractility reserve for the patient may be determined based on the multiple determinations of cardiac contractility.

In some embodiments, contractility reserve may be determined based, at least in part, by combining information from imaging (e.g., ultrasound) and a relationship between wall strain/wall motion and generated LV pressure as determined from a mechanical circulatory support device. In such embodiments, knowledge of the LV pressure waveform morphology coupled to strain rates, wall motion or end systolic volumes may be used to determine the contractility reserve of the patient.

It should be appreciated that one or more of the foregoing concepts may be combined in any suitable way to determine contractility reserve for a patient. For instance, in some embodiments, contractility reserve may be determined based, at least in part, on heart sound information, imaging information and information from an LV pressure waveform.

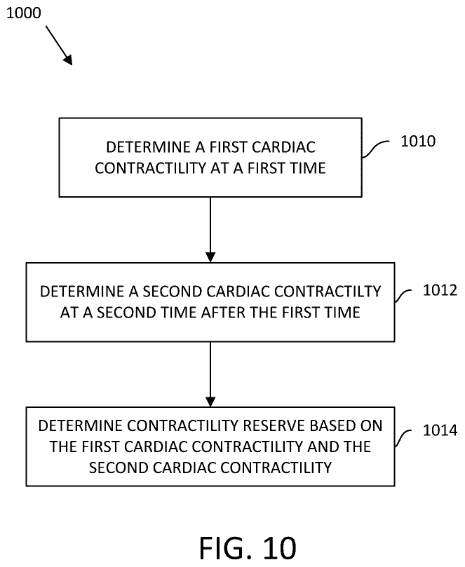

FIG. 10 illustrates a process 1000 for determining contractility reserve, in accordance with some embodiments of the present disclosure. Process 1000 may begin in act 1010, where a first cardiac contractility is determined based on signals recorded at a first time. For example, a set of features may be determined from signals recorded at a first time (e.g., a single point in time or a time window) from a mechanical circulatory support device, and the set of features may be provided as input to trained machine learning model trained, with the output of the trained machine learning model being the first cardiac contractility. Process 1000 may then proceed to act 1012, where a second cardiac contractility is determined based on signals recorded at a second time. For example, a set of features may be determined from signals recorded at a second time (e.g., a single point in time or a time window) from a mechanical circulatory support device, with the second time being after the first time. The set of features may be provided as input to trained machine learning model trained, with the output of the trained machine learning model being the second cardiac contractility. Process 1000 may then proceed to act 1014, where a contractility reserve is determined based on the first cardiac contractility and the second cardiac contractility. For example, the contractility reserve may be determined based on a ratio of the first and second cardiac contractility values, a difference between the first and second cardiac contractility values, a percent difference between the first and second cardiac contractility values, or a regression fit/projection of peak stress if multiple stress points are collected.

In some embodiments, the contractility reserve determined in act 1014 may be used to perform further actions including, but not limited to, providing clinical decision support to a healthcare provider, determining a weaning status or stability score for the patient, or other suitable actions described herein in connection with determining a measure of cardiac contractility.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation, many equivalents to the specific embodiments described herein. It is, therefore, to be understood that the foregoing embodiments are presented by way of example only and that inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein, unless clearly indicated to the contrary, should be understood to mean “at least one.”

The phrase “and/or,” as used herein, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

1. A computer-implemented method, comprising:
receiving a set of signals from a mechanical circulatory support device;
determining, using computer processor, a set of features based, at least in part, on the set of signals;
providing the set of features as input to a machine learning model trained to output a measure of cardiac contractility; and
performing an action based, at least in part, on the measure of cardiac contractility output by the machine learning model.

2. The computer-implemented method of claim 1, wherein the set of signals includes at least one pressure signal and/or at least one pump function signal.

3. The computer-implemented method of claim 2, wherein determining the set of features comprises determining at least one feature using a combination of a first pressure signal of the at least one pressure signal and a first pump function signal of the at least one pump function signal.

4. The computer-implemented method of claim 3, wherein the at least one feature using a combination of a first pressure signal of the at least one pressure signal and a first pump function signal of the at least one pump function signal comprises a maximum rate of rise of left ventricular pressure during contraction.

5. The computer-implemented method of claim 1, wherein determining a set of features comprises, determining one or more beat-to-beat features based, at least in part, on the set of signals.

6. The computer-implemented method of claim 5, wherein determining the one or more beat-to-beat features comprises determining one or more of mean pump flow, mean aortic pressure, left ventricle pressure at an end of diastole, a maximum rate of rise of left ventricular pressure during contraction, a mean motor current.

7. The computer-implemented method of claim 1, wherein the measure of cardiac contractility is an estimate of pre-load recruitable stroke work index.

8. The computer-implemented method of claim 1, wherein the machine learning model comprises a feedforward dense neural network.

9. The computer-implemented method of claim 1, wherein performing an action based, at least in part, on the measure of cardiac contractility output by the machine learning model comprises displaying an indication of the measure of cardiac contractility on a user interface associated with the mechanical circulatory support device.

10. The computer-implemented method of claim 9, wherein the indication of the measure of cardiac contractility is a trend of cardiac contractility over a particular time range.

11. The computer-implemented method of claim 1, wherein performing an action based, at least in part, on the measure of cardiac contractility output by the machine learning model comprises:
determining a stability score for a patient in which the mechanical circulatory support device is implanted, the stability score being based, at least in part, on the measure of cardiac contractility; and
displaying the stability score on a user interface associated with the mechanical circulatory support device.

12. The computer-implemented method of claim 1, wherein performing an action based, at least in part, on the measure of cardiac contractility output by the machine learning model comprises:
providing a treatment recommendation for a patient in which the mechanical circulatory support device is implanted, wherein the treatment recommendation is determined based, at least in part, on the measure of cardiac contractility.

13. The computer-implemented method of claim 1, wherein performing an action based, at least in part, on the measure of cardiac contractility output by the machine learning model comprises adjusting an operating condition of the mechanical circulatory support device.

14. The computer-implemented method of claim 13, wherein adjusting an operating condition of the mechanical circulatory support device comprises adjusting a pump speed of the mechanical circulatory support device.

15. The computer-implemented method of claim 1, wherein performing an action based, at least in part, on the measure of cardiac contractility output by the machine learning model comprises determining a contractility reserve based, at least in part, on the measure of cardiac contractility.

16. The computer-implemented method of claim 15, wherein
the measure of cardiac contractility includes a first cardiac contractility output from the machine learning model based on signals from the mechanical circulatory support device at a first time and second cardiac contractility output from the machine learning model based on signals from the mechanical circulatory support device at a second time after the first time, and
determining a contractility reserve comprises determining the contractility reserve based on the first cardiac contractility and the second cardiac contractility.

17. The computer-implemented method of claim 16, wherein determining the contractility reserve based on the first cardiac contractility and the second cardiac contractility comprises determining the contractility reserve based on a difference between the first cardiac contractility and the second cardiac contractility.

18. A controller for a mechanical circulatory support device, the controller comprising:
at least one hardware processor configured to:
determine a set of features based, at least in part, on a set of signals received from a mechanical circulatory support device;
providing the set of features as input to a machine learning model trained to output a measure of cardiac contractility; and
performing an action based, at least in part, on the measure of cardiac contractility output by the machine learning model.

19-34. (canceled)

35. A heart pump system, comprising:
a heart pump including at least one pressure sensor configured to sense a pressure within a portion of a heart of a patient; and
a controller configured to:
determine a set of features based, at least in part, on a set of signals received from heart pump, the set of features a first feature based on the sensed pressure;
provide the set of features as input to a machine learning model trained to output a measure of cardiac contractility; and
perform an action based, at least in part, on the measure of cardiac contractility output by the machine learning model.

36-44. (canceled)

45. The heart pump system of claim 35, further comprising:
a display configured to display a user interface including a representation of one or more signals associated with operation of the heart pump system,
wherein the controller is configured to perform an action based, at least in part, on the measure of cardiac contractility output by:
determining a stability score for a patient in which the heart pump is implanted, the stability score being based, at least in part, on the measure of cardiac contractility; and
displaying the stability score on the user interface.

46-51. (canceled)
